(19) United States
(12) Patent Application Publication
(10) Pub. No.: US 2008/0294188 A1
(43) Pub. Date: Nov. 27, 2008

(54) Implant Holder and Method of Deploying the Implant

(75) Inventors: William M. Appling, Granville, NY (US); William A. Cartier, Hampton, NY (US); Giorgio di Palma, Queensbury, NY (US)

Correspondence Address: Harry Ahn, Esq;Abelman, Frayne & Schwab, 666 Third Avenue, 10th Floor, New York, NY, 10017-5621, US

(73) Assignee: AngioDynamics, Inc., Queensbury, NY (US)

(21) Appl. No.: 11/752,716

(22) Filed: May 23, 2007

(51) Int. Cl. A61F 2/01

(52) U.S. Cl. 606/200

(57) ABSTRACT

An implant holder for use with a delivery catheter to deploy an implant, such as a filter, in a tubular body part is provided. The holder includes a cartridge body having a lumen extending between two ends and holding a collapsed implant, and an implant image disposed on the cartridge body. The implant image has an axial orientation corresponding to the axial orientation of the implant held in the cartridge body lumen. The axial orientation of the implant image helps to guide a physician in correctly orienting the implant regardless of which access point is used, thereby reducing the chance of deploying the implant in the wrong axial direction.

### FIELD OF THE INVENTION

The present invention relates to the field of medical devices. Particularly, the present invention pertains to a holder for an expandable implant and a method of using the holder for the purpose of delivering an expandable implant into a blood vessel in a patient's body.

### BACKGROUND OF THE INVENTION

Expandable medical implant devices are well known in the art. Such devices may include filters, stents, endografts, prosthetic valves such as venous valves, occluders, embolic coils, or any other type of expandable medical device with an asymmetrical configuration that can be delivered to a treatment point within a body vessel. Specifically, devices such as filters can be used in the vena cava, among other places. Such filters are often referred to as vena cava filters. Vena cava filters can have many different shapes or sizes, but typically, they have one or more sets of filtering legs that form a cone at one end of the filter that is open toward the direction of the blood flow. The filtering legs help to prevent large blood clots from passing through the filter, while permitting blood to flow through the filter. This helps prevent blood clots from reaching a patient's pulmonary arterial system, thereby alleviating potentially fatal consequences that could occur as a result of a pulmonary embolism (PE). The filtering legs can also have barbs or hooks on the ends of the filtering legs to help anchor the filter inside of the blood vessel. The filter may also have a hook for retrieval of the filter. For patients with deep vein thrombosis (DVT), or blood clotting within the deep venous system, the filter may be left in the vessel long-term, or the filter may be temporarily left in a patient, and thereafter retrieved.

Vena cava filters are most commonly placed in the inferior vena cava vein to capture clots originating from the lower extremities. The filter may be placed either below the renal veins (i.e., infra-renal placement) or above the renal veins (supra-renal placement). Vena cava filters may be placed in the superior vena cava vein. Although not as common, there is a growing trend toward placing filters in the superior vena cava due to the increase in use of long term vascular access devices such as central venous catheters and pacemakers. A common complication of these long term devices is upper-extremity DVT which may result in a potentially fatal PE. SVC filter placement can prevent PE associated with these long-term vascular access devices by capturing clots originating from the upper extremities.

Vena cava filters must be inserted into the blood vessel in the proper direction, i.e., the open end of the filter cone must be positioned upstream of the cone apex or hub so as to capture and retain clots within the cone. As blood flows through the open end of the cone, blood clots are caught inside of the filter cone and are pushed downstream into the center of the filter, where they are dissolved through the lysing action of the blood flow. Even non-conical filters may require a specific axial orientation within the vessel, due to anchoring barb orientation and/or retrieval hook position.

Filters placed in the inferior vena cava are typically inserted percutaneously via the femoral vein using the Seldinger technique. Access can also be achieved through the right internal jugular vein. This is advantageous in cases where there is thrombus in the iliac vein. Although the femoral and jugular approaches are most common, as medical devices and delivery systems become smaller, smaller vessels can be used as insertion sites, providing the physician with a broader range of access sites to choose from. Such alternative access sites may include, but are not limited to, brachial, antecubital, basilic, or subclavian venous access sites.

Because expandable implant devices, such as filters, may be inserted into blood vessels from different approaches, depending on the access site, it is critical for a physician to be able to insert an implant into the blood vessel with the proper axial orientation, i.e., with the open end of the filter cone positioned upstream of the apex. If the implant is placed in the wrong orientation in the blood vessel, the implant could be ineffective in capturing blood clots, which could put the patient at risk for pulmonary embolism. The wrong orientation of a filter could also result in localized thrombus build-up caused by turbulent blood flow around the mis-positioned filter. Localized thrombus build-up could lead to partial or complete vessel occlusion. An “upside-down” filter is also susceptible to migration because the anchoring elements, usually barbs or hooks, are typically designed to engage the vessel at a predetermined angle. If this angle is reversed, as would be the case in an incorrectly oriented filter, the anchors may not be able to retain the filter in place under a clot load, resulting in migration. This could lead to potentially fatal outcomes.

If the filter is inserted with the wrong orientation, the physician has two options to correct the error: retrieve the filter or leave the filter in place and insert another filter in the correct orientation. The physician may choose to simply place a second filter in the general vicinity of the first incorrectly oriented filter using the same access route. If the physician chooses to retrieve the mis-oriented filter, a second procedure is required to place a retrieval device through another insertion site in the patient's body. As an example, if the filter was incorrectly deployed using a jugular approach in the inferior vena cava, the retrieval hook would be positioned in a downstream direction, requiring a femoral retrieval approach. Both options may present additional complications associated with multiple insertion attempts and lengthened procedure time.

Placing a filter in the SVC with the correct orientation is even more critical because the “landing zone” of the SVC, or the segment defined by the SVC-right atrial junction and the confluence of the left and right brachiocephalic veins, is shorter than the IVC. This provides a relatively small area for safe filter deployment. Thus, it is not feasible to leave a filter in the SVC with the wrong orientation and to place another filter in the SVC. Instead, the filter must be retrieved prior to a second placement attempt. The location of the parietal pericardium relative to the SVC is also very variable. If the filter legs push through the SVC and penetrate the pericardium, it may cause tamponade and heart failure. Thus, it is critical to ensure that the filtering leg anchoring mechanisms are located in the proper orientation so as to reduce the risk of penetration.

Expandable medical implant devices, such as filters, are typically deployed using catheter-based delivery systems. Such delivery systems may optionally include a cartridge or other holder containing the implant in a collapsed position. Cartridges are pre-loaded with the implant in a pre-determined orientation by the manufacturer. Such cartridges may also have arrows or words marked on the outside of the cartridge, which indicate the orientation of the expandable implant inside the cartridge. Cartridges typically have two ends, one of which is attached to a delivery catheter. A pusher wire is then inserted into the cartridge through lumen. The pusher is used to advance the collapsed implant device through the catheter lumen, and into the blood vessel.

Several cartridges have been proposed to assist physicians in determining the correct orientation of expandable implant devices, such as filters. Such cartridges have different arrows, labels and/or indicators located on the cartridge, indicating “femoral” or “jugular,” corresponding to the desired orientation. These indicia show which end of the cartridge to insert first into a catheter to assist in proper orientation of the filter during delivery and treatment. Such filter cartridges may also have different shaped ends which can be connected to the catheter, depending on whether the femoral or jugular access sites are chosen. For instance, one end may be a square shape and the other may be a triangle or circle shape.

The use of pre-loaded filter cartridges is advantageous because it reduces the inventory a hospital is required to carry. Since the physician determines the orientation of the cartridge relative to the catheter, a single delivery system is stocked for either the femoral or jugular approach. Although the use of attachable cartridges has reduced the inventory hospitals are required to carry, there is an increased risk that the physician might make an error when attaching the cartridge to the catheter, causing the filter to be deployed in the wrong orientation, as described above.

Medical filter cartridges also have other disadvantages. The markings on current filter cartridges may not provide indicia for insertion into alternate access sites, other than the femoral or the jugular approaches. In addition, these cartridges cannot be used to indicate correct orientation of the device when placed in the superior vena cava. The blood flow in the SVC is opposite that of the IVC. Thus, to maintain the correct orientation of the filter in the SVC, a filter designed for jugular access must be placed via a femoral approach, whereas a filter designed for femoral access must be placed via a jugular approach. Any filter orientation indicator that uses arrows or text labeling cannot be applied to the SVC without placing the filter in the opposite direction from the indicator direction.

Although the presence of arrows pointing in certain directions or different shaped ends of the filter allegedly aid the physician in figuring out which end of the cartridge to attach to the catheter, the physician may still not know whether the pre-loaded filter that is inside of the cartridge is in the proper orientation. Filter deployment errors are often due to operator error while using a filter delivery mechanism. The physician may mistake the orientation of the filter by misinterpreting the arrows, colors, or other indicia on the cartridges. The Food and Drug Administration (FDA) Manufacturer and User Facility Device Experience (MAUDE) database contains numerous reports involving users deploying filters with the wrong orientation using cartridges with directional indicators. Specifically, it was reported recently that an operator misunderstood the meaning of the arrows and labels on a Cordis OptEase® vena cava filter cartridge and deployed the filter in the wrong direction.

All of the currently proposed cartridge designs present the risk that the physician will still attach the delivery catheter to the wrong end of the cartridge, which could result in the deployment of the filter inside of the blood vessel in the wrong orientation. Thus, there has been, and continues to be, a need for a solution to the above mentioned problems, such as a device and method which allows a physician to reliably and accurately determine the orientation of an expandable implant device that is located inside of a cartridge, before attaching the pre-loaded implant cartridge to a delivery catheter and deploying the implant inside of a blood vessel.

### SUMMARY OF THE INVENTION

An implant device holder for use with a delivery catheter to deploy an implant device in a tubular body part is provided. The holder includes a cartridge body having a lumen extending between two ends, holding a collapsed implant device, and displaying an implant image. The implant image has an axial orientation corresponding to the axial orientation of the implant held in the cartridge body lumen. The axial orientation of the implant image helps to guide a physician in correctly orienting the implant regardless of which access point is used, thereby reducing the chance of deploying the implant in the wrong axial direction.

According to another aspect of the disclosure, a method of deploying an implant device in a tubular body part using an implant holder is provided. The holder has a cartridge body having a lumen extending between two ends and an implant image that has an axial orientation corresponding to the axial orientation of the implant held in the cartridge body lumen. A delivery catheter is inserted into a tubular body part through an access site. Based on the axial orientation of the implant image disposed on the cartridge body and the location of the access site, the implant cartridge body is oriented such that either one or the other end of the cartridge body faces a proximal end of the delivery catheter. The oriented cartridge body is then attached to the proximal end of the delivery catheter. The implant device contained in the attached cartridge body is moved in a distal direction so as to deploy the implant.

### BRIEF DESCRIPTION OF THE DRAWINGS

FIG. 1A is a plan view of an implant device holder, according to the present invention.

FIG. 1B is an end view of the implant device holder of FIG. 1A, according to the present invention.

FIG. 1C is a side view of the implant device holder of FIG. 1A, according to the present invention.

FIG. 1D is a cross-sectional view of the implant holder, as seen along line 1D-1D in FIG. 1C, according to the present invention.

FIG. 1E is a plan view of another embodiment of the implant holder, according to the present invention.

FIG. 1F is a plan view of another embodiment of the medical implant holder, according to the present invention.

FIG. 1G is a plan view of another embodiment of the implant holder, according to the present invention.

FIG. 2 illustrates an implant delivery system for deploying an implant into a chosen access site, according to the present invention.

FIG. 3 illustrates a delivery catheter which has been advanced to a desired location from a chosen access site for implant deployment, according to the present invention.

FIG. 4 illustrates the delivery catheter of FIG. 3 to which an implant holder is securely attached, according to the present invention.

FIG. 5 illustrates a collapsed implant being pushed through the delivery catheter by a pusher of the implant delivery system, according to the present invention.

FIG. 6 illustrates the implant device that has been deployed by the implant delivery system, according to the present invention.

FIG. 7 illustrates the delivery catheter which has been advanced to a desired location from a different access site, according to the present invention.

FIG. 8 illustrates the delivery catheter of FIG. 7 to which an implant holder is securely attached, according to the present invention.

FIG. 9 illustrates a collapsed filter being pushed through the delivery catheter by a pusher of the implant delivery system, according to the present invention.

FIG. 10 illustrates the implant that has been deployed by the implant delivery system, according to the present invention.

### DETAILED DESCRIPTION OF THE INVENTION

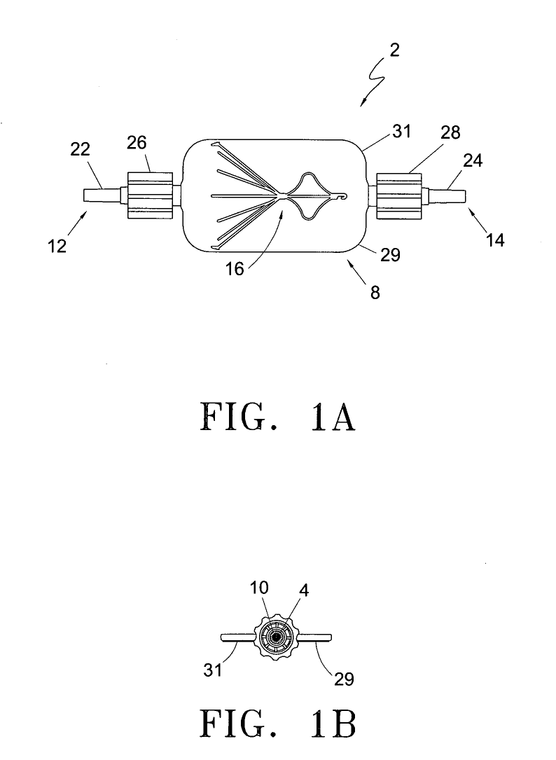

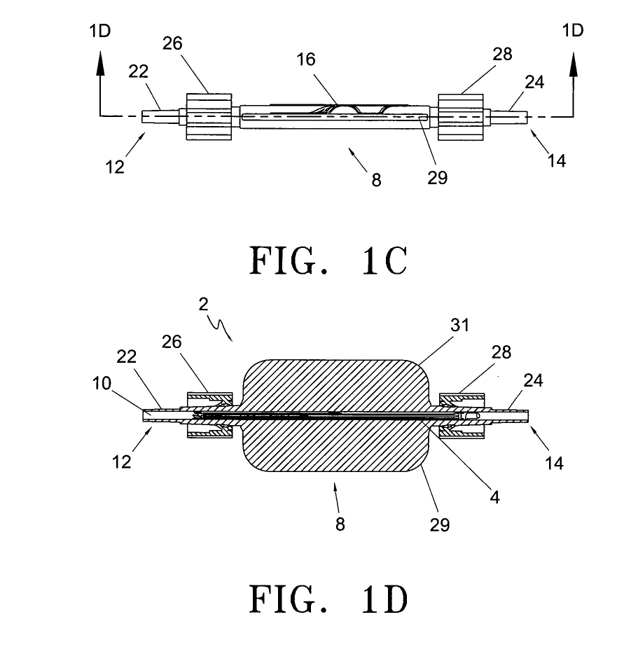

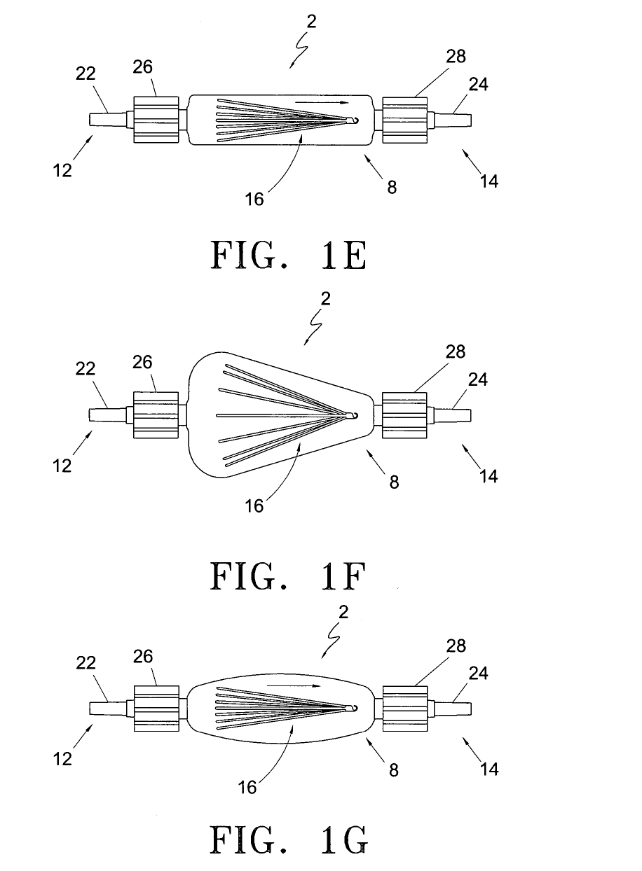

FIGS. 1A-1G illustrate an implant holder/cartridge 2 for use with a delivery catheter 32 for deploying an implant device 4 in a blood vessel 6. The holder 2 includes an implant device cartridge body 8 having a lumen 10 extending between first and second longitudinal ends 12, 14. The lumen 10 holds the implant device 4 in a radially collapsed state (see FIG. 1D). Although a filter device is described herein, any type of expandable medical implant device 4 may be used, such as, but not limited to, stents, endografts, prosthetic valves such as venous valves, occluders, embolic coils, or any other type of expandable medical device with an asymmetrical configuration or that requires a specific axial orientation and can be delivered to a treatment point within a body vessel 6. Such devices may be self-expandable or may be expandable by a mechanical means. One of ordinary skill would recognize that there are potentially an infinite amount of shapes and designs that may be chosen for filters or other expandable medical implant devices that can be used in the present invention, as illustrated in FIGS. 1A through 1G.

According to the invention, an image 16 of the expandable implant 4 is disposed on the cartridge body 8. Preferably, the image 16 represents the implant device 4 in a fully expanded state and has an axial orientation corresponding to the axial orientation of the implant device 4 held in the cartridge body lumen 10. However, the image 16 could be in a partially expanded or even collapsed state so long as the axial orientation of the implant image 16 can be ascertainable by a physician and that it corresponds to the axial orientation of the implant device 4. In one implementation, a thin self-adhesive film containing the implant image 16 is attached to an outer surface of the cartridge body 8. The implant image 16 may also be in the form of a printed image, an embossed label, a raised imprint on the cartridge body 8 that is produced by injection molding and is part of the cartridge body 8, or any other suitable permanent form. Alternatively, the implant image 16 may be the filter cartridge body 8 itself. The implant image 16 may be made of any suitable color or design, including clear or transparent. The implant image 16 may be disposed anywhere on the cartridge body 8, including any inner or outer surface of the cartridge body 8.

The expanded implant image 16 takes up a larger surface area than the collapsed implant 4. The cartridge body 8 has a pair of radially extended wings 29, 31 over which the implant image 16 extends. Advantageously, the implant image 16 feature allows a physician to visually check how and in which direction the collapsed implant device 4 in the lumen 10 will be deployed into the expanded state inside the blood vessel 6, as will be discussed more fully later herein. In another embodiment, the cartridge body may optionally have indicators in addition to or in place of the implant image 16, such as, but not limited to, arrows, or other markers, indicating the direction of blood flow relative to the implant device 4 (as shown in FIGS. 1E and 1G).

The cartridge body 8 includes first and second luer connectors 26, 28 that securely connect the implant device holder 2 to the delivery catheter 32. The cartridge body 8 also includes first and second hollow extensions 22, 24, respectively at the first and second ends 12, 14. Each extension 22, 24 is adapted to open a hemostasis valve of the delivery catheter 32. The valve is used to prevent leakage of fluids through the proximal end of the delivery catheter 32. The extensions 22, 24 also serve to prevent a standard luer connectable device from being erroneously connected to the luer connectors 26, 28, thereby acting as a safety feature. The extensions 22, 24 thus help to prevent the cartridge body 8 from being connected to the wrong catheter.

Between the extensions 22, 24 is a single through lumen 10 that extends through the center of the cartridge body 8 and is in communication with the catheter 32. The length and diameter of the through lumen 10 can be of any suitable dimensions, depending on the type of expandable implant device 4 used. Although the cartridge body 8 is illustrated herein as having a square or semi-rectangular shape, the cartridge body 8 may have any suitable shape. Such shapes may include, but are not limited to, a round cylindrical shape like a tube design, as illustrated in FIG. 1E, a tapered triangular shape or a similar shape as the implant device 4 itself, as illustrated in FIG. 1F, or an oval shape, as indicated in FIG. 1G, to name a few. The cartridge body 8 of the present invention may be made from any plastic medical-grade material that is well known for such use and may be made of any suitable color, including a clear or transparent design, thereby allowing visualization of a pre-loaded expandable implant device 4.

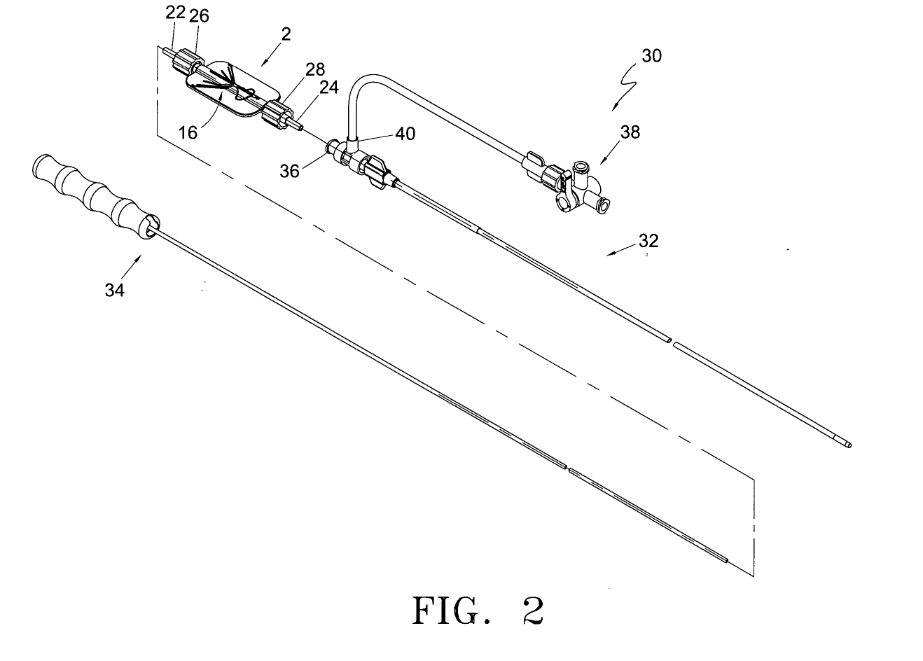

The expandable implant device 4 contained in the holder 2 is deployed using a delivery system 30 as shown in FIG. 2. Although the delivery system 30 described herein includes a 6F sheath, which is compatible with an 0.035″ guidewire, other size sheaths may be used. The system 30 includes the holder 2 which houses the implant 4, delivery catheter 32, and a pusher 34. The pusher 34 may be reinforced by a wire. The delivery catheter 32 includes a luer connector 36 for mating and locking with the luer connector 26 or 28 depending on the axial orientation of the cartridge body 8. Procedural fluids may be flushed through a lumen of the delivery catheter 32 through a side arm stopcock assembly 38 or through the cartridge body 8. The sidearm stopcock assembly 38 can also be used to administer saline or emergency drugs directly into the blood vessel 6.

Thus, in a key advantage of this invention, the risk of deploying a filter or other expandable implantable device 4 in the wrong orientation is minimized. The physician is not required to interpret indicators such as arrows or words alone, but instead is presented with an implant image 16 clearly depicting the final orientation of the expanded device 4 when deployed, making the decision of which end of the holder 2 to attach to the catheter 32 clear to the physician. In addition, the physician is not restricted to femoral or jugular approaches, but may utilize this invention to deploy an asymmetrical implant 4 from any desired access approach, as the physician can reliably and accurately determine the correct orientation of the implant 4 for deployment in the vessel 6 with differing blood flow patterns, such as in the upper versus lower extremity vasculature.

Another advantage of the present invention is that it provides one easy to use, low-cost, single kit for physicians and hospitals, which may be used with any access site in a patient's body. This eliminates the need for physicians and hospitals to carry an inventory of different pre-loaded delivery systems that are specific to either the femoral or the jugular approaches. The implant image 16 on the cartridge body 8 is also advantageous in that it serves as a quality control mechanism to ensure that manufacturing personnel will pre-load the filter 4 into the cartridge body 8 in the proper orientation. The implant image 16 provides a convenient visual identifier for manufacturing personnel, thereby allowing the expandable implant device 4 to be more accurately and reliably loaded into the holder 2 in the correct orientation.

A procedure for deploying the expandable implant device 4 into a blood vessel 6 will now be described in detail. Although the femoral vein access approach will be described herein, any desired access point may be used, such as, but not limited to, jugular, brachial, antecubital, basilic, or subclavian venous access sites. The expandable implant device 4 may be deployed inside any tubular body part, including ducts. The blood vessel 6 may be, but is not limited to, the inferior or superior vena cavae.

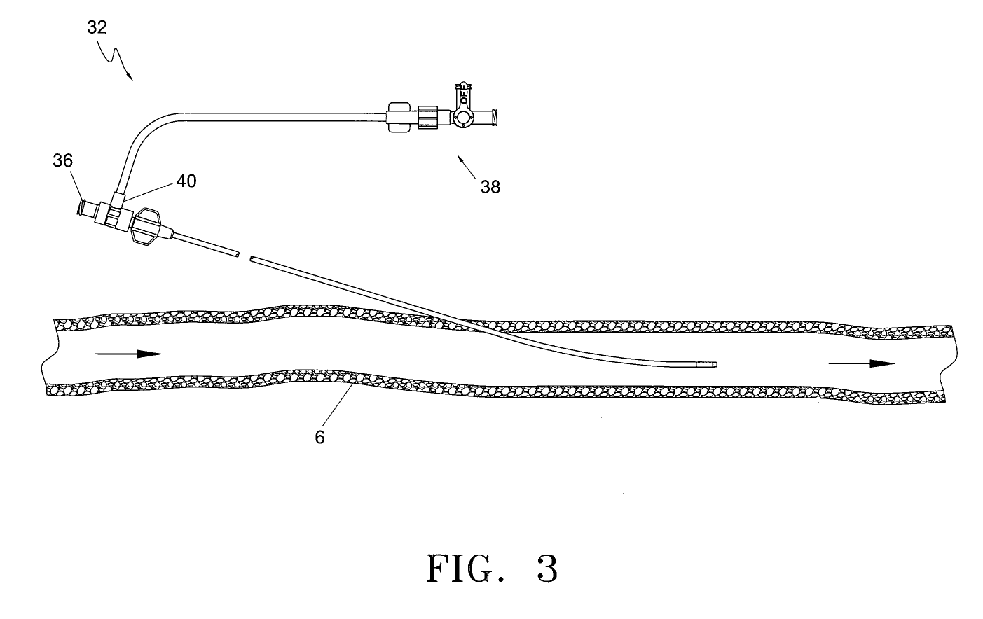

FIGS. 3-7 depict a filter 4 deployment using a femoral vein approach. The direction of blood flow is depicted by the arrows inside of the blood vessel 6. The deployment procedure begins with a standard pre-operative preparation of the patient as is well known in the art. Access to the femoral vein is gained using a standard Seldinger technique. A small gauge needle is used to puncture the skin and access the vein. A guidewire is advanced into the vein through the lumen of the needle. The needle is then removed, leaving the guidewire in place. The delivery catheter 32 is then advanced over the guidewire until the distal tip is placed at a desired location (e.g., inferior vena cava).

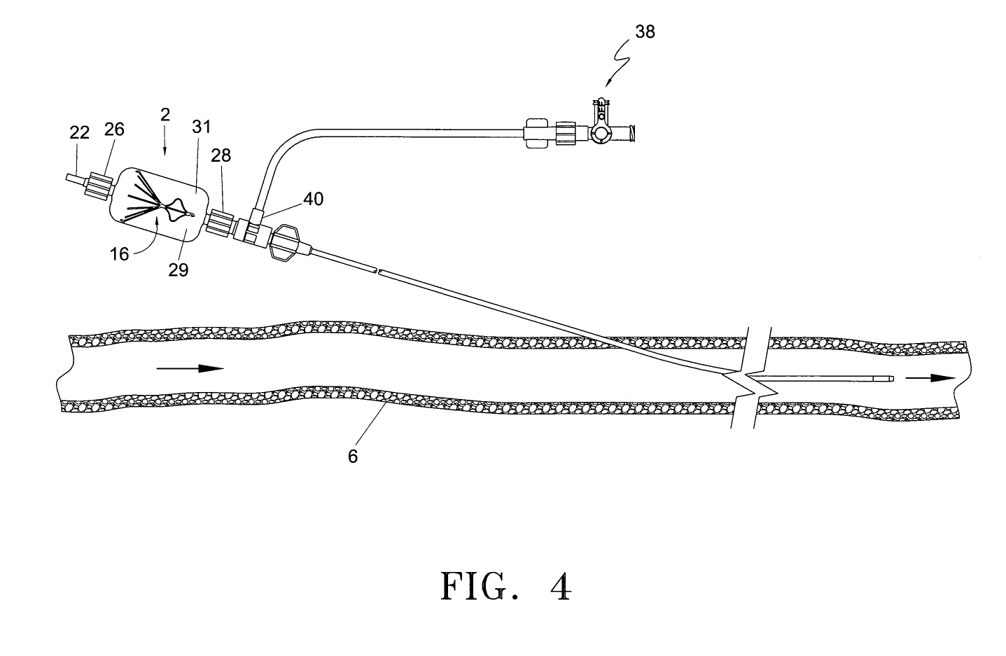

Once the delivery catheter 32 is in place, the physician uses the implant image 16 on the cartridge holder body 8 to determine the correct axial orientation of the collapsed filter 4. For a femoral approach, the physician orients the cartridge holder 2 with the luer connector 28 facing the proximal end of the delivery catheter 32. This orientation positions the conical apex of the filter 4 in the downstream direction, as illustrated by the arrows, once deployed. Once properly oriented, the expandable implant device holder 2 is securely attached to the proximal end of the delivery catheter 32, as shown in FIG. 4. Specifically, the second extension 24 of the filter cartridge body 8 is pushed into the lumen of the delivery catheter 32 as the luer lock connector 28 is connected to the luer lock connector 36 (shown in FIG. 3) so as to securely attach the holder 2 to the delivery catheter 32. This causes a hemostasis valve located near the proximal end of the catheter 32 to open. The hemostasis valve has a rubber ring that surrounds the inside of the valve. When the extension 24 is inserted into the hemostasis valve, depending on which end is used, the extension 24 expands the inside of the valve and moves the gasket inside of the hemostasis valve from a closed to an open position. The filter 4 is protected within the extension 24 lumen, preventing the legs and barbs of the filter 4 from catching on the inside wall of the valve as the filter 4 is advanced through the delivery catheter 32.

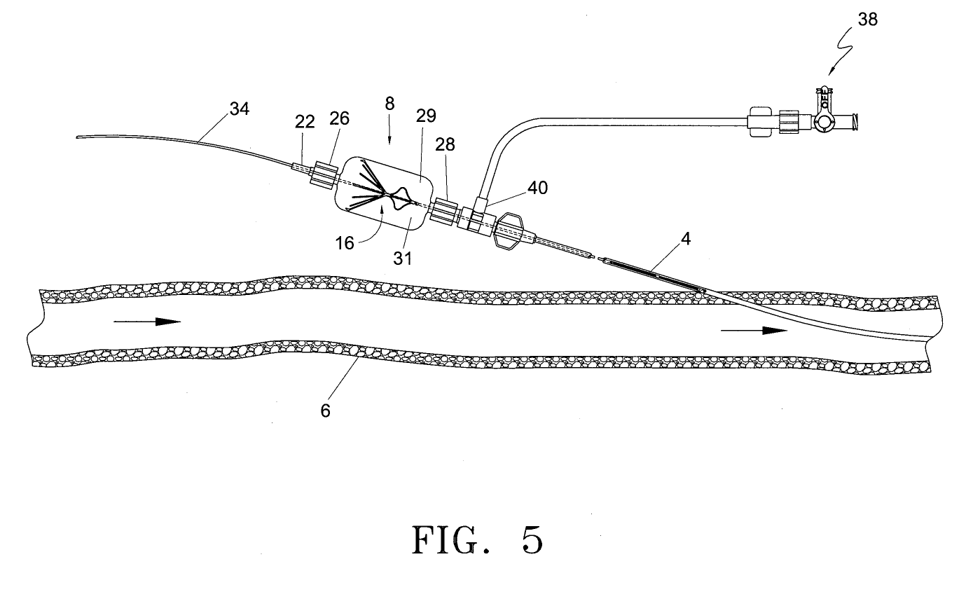

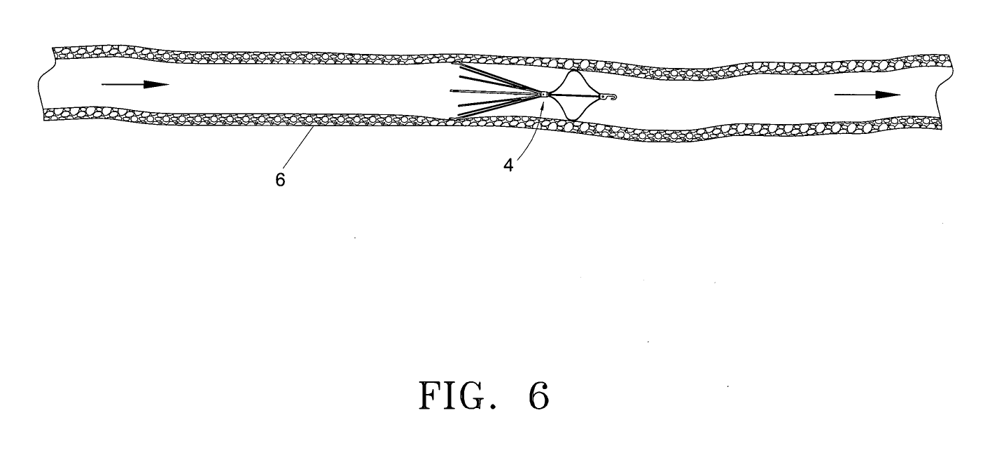

The pusher 34 is next inserted into the cartridge body 8 through the first extension 22, as shown in FIG. 5, so that the distal end of the pusher 34 moves the filter 4 through the delivery catheter 32 in a distal direction until the filter 4 is pushed through the distal end of catheter 32. Alternatively, the filter 4 may be advanced by the pusher 34 until the filter 4 is positioned near the distal end of the catheter 32. Then the catheter 32 can be retracted while holding the pusher 34 stationary. The filter 4 expands as it is unsheathed by the retraction of the catheter 32. Once the filter 4 exits from the delivery catheter 32, it is no longer constrained and expands into its fully expanded state as shown in FIG. 6. Following expansion of the filter 4, the entire delivery system 30 is withdrawn, and deployment of the filter 4 is complete.

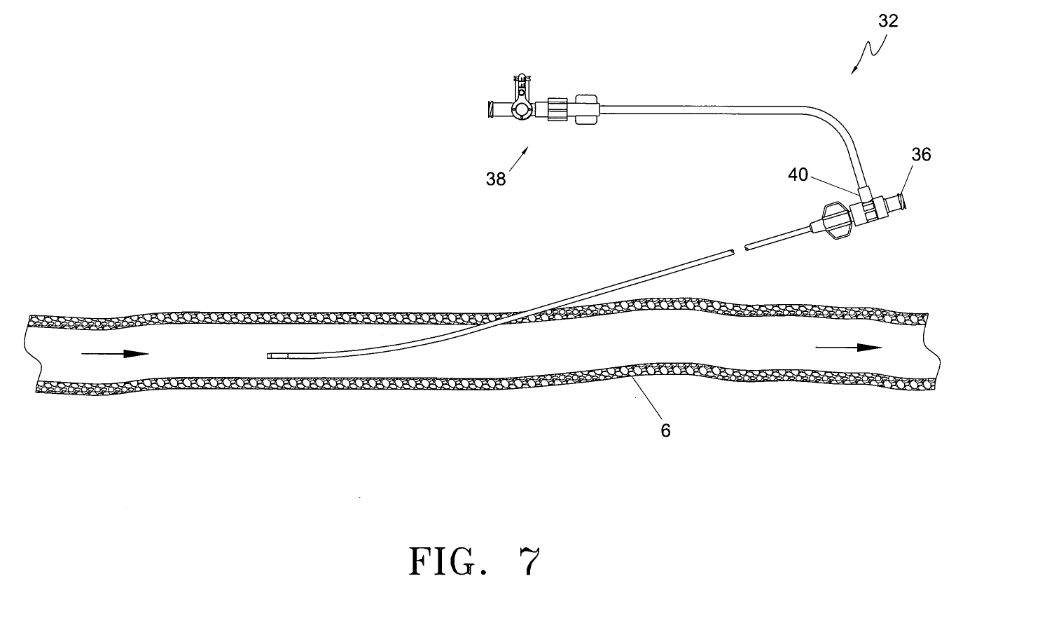

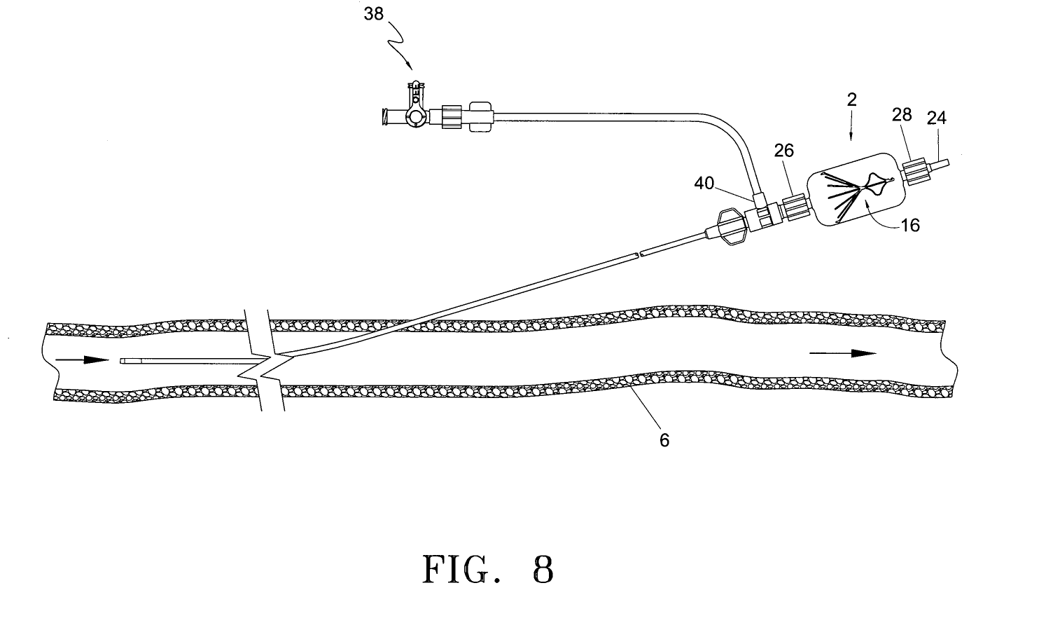

Referring now to FIGS. 7-10, the implant holder 2 of the current invention is used to deploy a filter 4 in the blood vessel 6 using a jugular approach. FIG. 7 depicts the delivery catheter 32 after insertion and positioning within the blood vessel 6. Once the delivery catheter 32 is in place, the physician uses implant image 16 on the cartridge holder body 8 to determine the correct axial orientation of the collapsed filter 4. The physician orients the cartridge holder 2 with the luer connector 26 facing the proximal end of the delivery catheter 32. This orientation positions the conical apex of the filter 4 in the downstream direction (illustrated by the arrows). The physician then securely attaches the expandable implant device holder 2 to the delivery catheter 32 (as shown in FIG. 8) by attaching the luer lock connector 26 to the luer lock connector 36 and inserting first extension 22 of the filter holder 2 into the lumen of the delivery catheter 32, which causes the hemostasis valve of the catheter 32 to open, as described above.

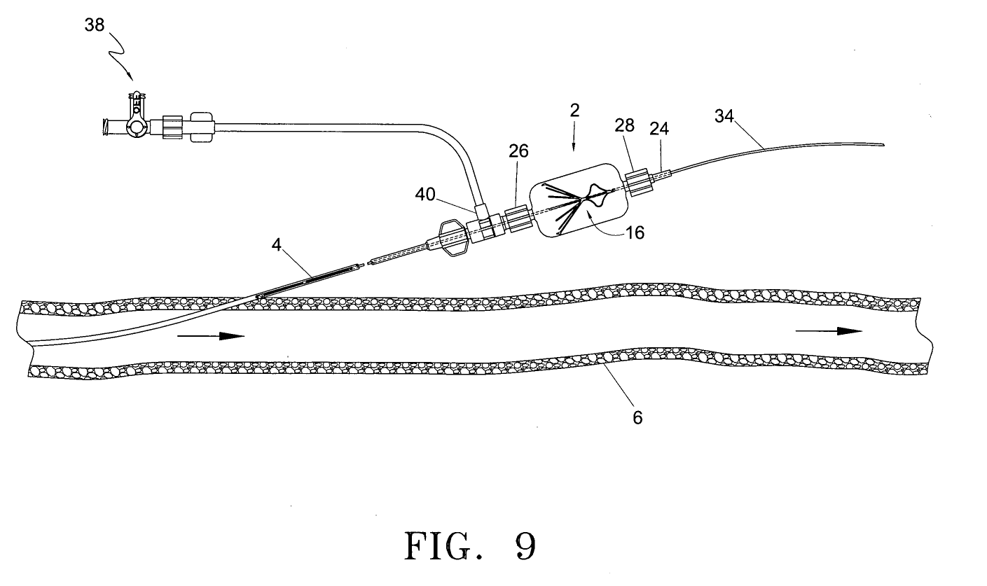

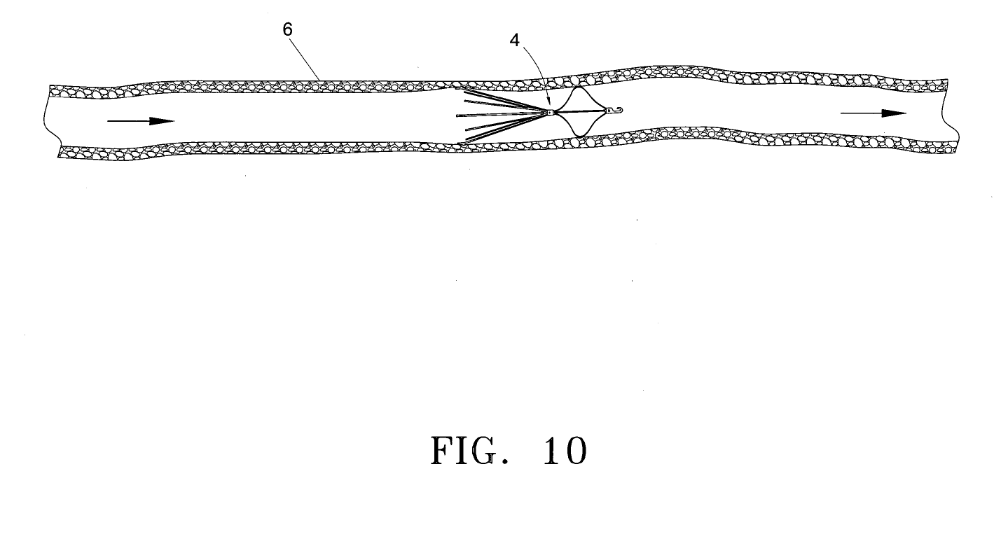

To deploy the filter 4 within the vessel 6, the pusher wire 34 is inserted through lumen 10 of holder 2, as shown in FIG. 9, and advanced until the filter 2 is positioned near the distal end of the delivery catheter 32. The catheter 32 is then retracted while holding the pusher 34 stationary, which causes the filter 4 to be deployed into the vessel 6. No longer constrained by the catheter 32 walls, the filter 4 expands to its fully expanded configuration as shown in FIG. 10. Unlike the femoral approach in which the apex or head of the filter 4 is deployed through the delivery catheter 32 first, for a jugular delivery, the filter 4 legs are deployed first.

As discussed above, the axial orientation of the implant image 16 corresponds to the axial orientation of the implant device 4 held in the cartridge lumen 10 to clearly show which direction the implant device 4 would deploy. This feature helps to guide the physician in correctly orienting the filter 4 regardless of which access point is used, thereby reducing the chance of deploying the filter 4 in the wrong axial direction. Although the method described herein covers deployment of a filter 4 in the blood vessel 6 using femoral and jugular approaches, other tubular body parts and approaches are within the scope of this invention.

The method disclosed herein may be used to deploy single expandable implants 4 or multiple expandable implant devices 4, of the same or different types, depending on the type of treatment and access site chosen. In the case of multiple implants 4, multiple implant images 16 may be used on the cartridge body 8 to aid a physician with the correct orientation of the filter 4, provided that the implant images 16 convey the correct axial orientation of the corresponding implants 4.

The present invention also encompasses a kit to be used with the method of the present invention. The kit may include, but is not limited to, the delivery catheter 32, implant holder/cartridge 2, at least one expandable implant device 4 that may be pre-loaded in the implant holder 2, and pusher 34.

The foregoing specific embodiments represent just some of the ways of practicing the present invention. Many other embodiments are possible within the spirit of the invention. Accordingly, the scope of the invention is not limited to the foregoing specification, but instead is given by the appended claims along with their full range of equivalents.

What is claimed is:

1. An implant holder for use with a delivery catheter to deploy an expandable implant in a tubular body part, the holder comprising:
a cartridge body having a lumen extending between a first end and a second end and being adapted to hold the implant in a collapsed state;
the cartridge body displaying an implant image having an axial orientation corresponding to the axial orientation of the implant held in the cartridge body lumen.

2. The holder according to claim 1, wherein the cartridge body lumen is adapted to hold the implant having an asymmetrical deployment configuration.

3. The holder according to claim 1, wherein the cartridge body lumen is adapted to hold a self-expandable implant.

4. The holder according to claim 1, wherein the cartridge body lumen is adapted to hold the implant that is deployable in a blood vessel.

5. The holder according to claim 1, wherein the cartridge body lumen is adapted to hold a filter.

6. The holder according to claim 1, wherein the shape of the cartridge body represents the implant image.

7. The holder according to claim 1, wherein the implant image is disposed on or within the cartridge body.

8. The holder according to claim 1, wherein the implant image represents the implant in an expanded state.

9. The holder according to claim 1, wherein the cartridge body comprises first and second radially extended wings, and the implant image extends over the wings.

10. The holder according to claim 1, wherein the cartridge body comprises first and second extensions disposed at the respective first and second ends of the cartridge body.

11. The holder according to claim 10, wherein the first and second extensions are adapted to open a valve of the delivery catheter.

12. The holder according to claim 10, wherein the cartridge body comprises first and second connectors disposed at the respective first and second ends of the cartridge body with each connector adapted to securely connect the cartridge body to the delivery catheter.

13. The holder according to claim 1, wherein the cartridge body comprises first and second connectors disposed at the respective first and second ends of the cartridge body with each connector adapted to securely connect the cartridge body to the delivery catheter.

14. The holder according to claim 1, wherein the cartridge body also displays an indicator representing the direction of blood flow relative to the implant.

15. An implant holder for use with a delivery catheter for deploying an expandable implant in a tubular body part, the holder comprising:
an expandable implant;
a cartridge body having a first end, a second end opposite to the first end and a lumen, the lumen extending between the first and second ends and holding the expandable implant in a collapsed state;
the cartridge body displaying an implant image representing the implant in an expanded state and having an axial orientation corresponding to the axial orientation of the implant held in the cartridge body lumen.

16. The holder according to claim 15, wherein the cartridge body lumen is adapted to hold the implant having an asymmetrical deployment configuration.

17. The holder according to claim 15, wherein the cartridge body lumen is adapted to hold a self-expandable implant.

18. The holder according to claim 15, wherein the cartridge body lumen is adapted to hold the implant that is deployable in a blood vessel.

19. The holder according to claim 15, wherein the cartridge body lumen is adapted to hold a filter.

20. The holder according to claim 15, wherein the shape of the cartridge body represents the implant image.

21. The holder according to claim 15, wherein the implant image is disposed on or within the cartridge body.

22. The holder according to claim 15, wherein the cartridge body comprises first and second radially extended wings, and the implant image extends over the wings.

23. The holder according to claim 15, wherein the cartridge body comprises first and second extensions disposed at the respective first and second ends of the cartridge.

24. The holder according to claim 23, wherein the first and second extensions are adapted to open a valve of the delivery catheter.

25. The holder according to claim 23, wherein the cartridge body comprises first and second connectors disposed at the respective first and second ends of the cartridge body with each connector adapted to securely connect the cartridge body to the delivery catheter.

26. The holder according to claim 15, wherein the cartridge body comprises first and second connectors disposed at the respective first and second ends of the cartridge body with each connector adapted to securely connect the cartridge body to the delivery catheter.

27. A method of deploying an expandable implant in a tubular body part using an implant holder having a cartridge body having a lumen extending between a first end and a second end and being adapted to hold the implant in a collapsed state, the cartridge body displaying an implant image having an axial orientation corresponding to the axial orientation of the implant held in the cartridge body lumen, the method comprising:
inserting a delivery catheter into the tubular body part through an access site;
orienting the cartridge body based on the axial orientation of the implant image and the location of the access site such that either the first end or the second end faces a proximal end of the delivery catheter;
attaching the oriented cartridge body to the proximal end of the delivery catheter; and
moving the expandable implant contained in the cartridge body lumen through the delivery catheter in a distal direction so as to deploy the expandable implant.

28. The method according to claim 27, wherein:
the cartridge body comprises first and second extensions disposed at the respective first and second ends of the cartridge body; and
the step of attaching includes pushing the first or second extension through a valve disposed at the proximal end of the delivery catheter to open the valve.

29. The method according to claim 27, wherein the cartridge body further comprises first and second connectors disposed at the respective first and second ends of the cartridge body and wherein the step of attaching includes connecting the first or second connector with a complementary connector disposed at the proximal end of the delivery catheter to securely connect the cartridge body to the delivery catheter.

30. The method according to claim 27, wherein:
the expandable implant is a filter; and
the step of moving the expandable implant includes moving the expandable filter contained in the cartridge body lumen through the delivery catheter in a distal direction so as to deploy the expandable filter.

31. The method according to claim 27, wherein the step of moving the expandable implant includes pushing the implant through the delivery catheter.

32. A kit comprising:
a delivery catheter;
an implant holder for use with the delivery catheter to deploy an expandable implant in a tubular body part, wherein the holder comprises a cartridge body having a lumen extending between a first end and a second end and being adapted to hold the implant in a collapsed state, the cartridge body displaying an implant image having an axial orientation corresponding to the axial orientation of the implant held in the cartridge body lumen;
at least one expandable implant; and
a pusher.
